(19) United States
(12) Patent Application Publication
(10) Pub. No.: US 2017/0035389 A1
(43) Pub. Date: Feb. 9, 2017

(54) A VETERINARY GLOVE, A VETERINARY IMPLEMENT FOR RECTAL ULTRASOUND EXAMINATION OF A LARGE ANIMAL, AND A METHOD OF PREPARING SUCH AN IMPLEMENT

(71) Applicant: Jørgen Kruuse A/S, Langeskov (DK)

(72) Inventor: Keith E. Sterner, Ionia, MI (US)

(73) Assignee: Jørgen Kruuse A/S, Langeskov (DK)

(21) Appl. No.: 15/302,926

(22) PCT Filed: Apr. 7, 2015
(86) PCT No.: PCT/DK2015/050081

§ 371 Date: Oct. 7, 2016

(30) Foreign Application Priority Data
Apr. 10, 2014 (DK) PA 2014 70206

(51) Int. Cl. A61B 8/00; A61B 90/53; A61B 8/12; A61B 42/10; A41D 19/00; A61B 5/00

(52) U.S. Cl. CPC A61B 8/4455; A41D 19/0027; A41D 19/0075; A61B 5/6806; A61B 8/12; A61B 8/445; A61B 8/4281; A61B 42/10; A41D 19/0037; A61B 90/53; A61B 2503/40

(57) ABSTRACT

A veterinary glove (9) for use in rectal ultrasound examination of a large animal by means of an ultrasound transducer. The glove (9) has a fluid tight glove wall (15) defining a hand receiving glove body (10) for receiving a hand of a person performing the examination together with the ultrasound transducer. The glove (9) comprises a palm portion (12) integral with a thumb finger pocket (18a), an index finger pocket (18b) and one or more additional finger pockets (18c, 18d, 18e), and the glove wall (15) further defines an ultrasound transducer pocket (22) integral with the palm portion (12) and communicating with the inside of the glove (9), the elongated transducer pocket (22) extends either i) between the thumb finger pocket (18a) and the index finger pocket (18b) or ii) being arrangeable to extend between the thumb finger pocket (18a) and the index finger pocket (18b).

### FIELD OF THE INVENTION

The present invention relates generally to a veterinary glove for use in the rectal ultrasound examination of a large animal, such as for bovine or equestrian rectal examination, by means of an ultrasound transducer.

Such rectal examinations may inter alia be performed for pregnancy diagnosis or for assessing ovarian structures, and the transducers may, by way of example only, be 5 MHz linear transducers producing ultrasound waves that are reflected in part; the reflected waves or echoes are displayed on a screen. Rectal ultrasonography where an ultrasound transducer is inserted into the rectal canal of the animal has for many purposes replaced palpation by hand in that the errors may be reduced.

### BACKGROUND OF THE INVENTION

In performing rectal ultrasound examinations sometimes a generally elongated transducer with a handle will be mounted to the end of an elongated inserter, or placed in a sheath, which is then inserted into the rectal canal by a veterinarian wearing a glove.

However, in most instances the veterinarian will take on an examination glove, hold on to the transducer handle with his gloved hand, and then insert his hand and arm into the rectal canal while holding securely on to the transducer; this allows the veterinarian to obtain a direct feel of the rectal canal with his fingers. Immediately before this step the veterinarian may have emptied the rectum using his hand, wearing the same glove. Examination gloves for this purpose are commonly available, and often have a sleeve-like extension running up to the shoulder of the veterinarian; an example is shown in KR 20120002725. Another example of a glove for veterinarian use is shown in GB 2484105.

CN 201995640, FR 1398821, U.S. Pat. No. 2,661,476 and CN 203089825 disclose work gloves which, however, are not suitable or intended for veterinary, let alone rectal, examinations. Examples of medical gloves are disclosed in U.S. Pat. No. 7,832,021, U.S. Pat. No. 6,409,688 and DE 1038712. In DE 1038712 a stethoscope is placed with its sound capturing part inside a finger pocket which has a bayonet socket and which, hence, is not fluid tight.

EP 0173837 discloses a disposable arm-long rubber pouch for receiving an ultrasound transducer together with the hand and arm of a veterinarian or other person for performing rectal ultrasound examination of a large animal.

It is a problem with the aforementioned known procedure that holding securely on to the transducer with a gloved hand during the examination procedure causes additional strain to the veterinarian who already is in a working position causing neck and shoulder pain due to repetitive movements of his neck and arm.

### SUMMARY OF THE INVENTION

Accordingly, it is an object of the present invention to improve working conditions for the veterinarian. This is achieved by the examination glove and implement defined in the claims.

The invention also permits the veterinarian to sanitize the transducer only after conclusion of several consecutive examination procedures; in conventional uses the transducer becomes contaminated by manure in the rectal canal, requiring the veterinarian to sanitize the transducer before examining another animal.

More particularly, the present invention proposes a veterinary glove for use in rectal ultrasound examination of a large animal by means of an ultrasound transducer. The glove has a fluid tight glove wall defining a hand receiving glove body for receiving a hand of a person performing the examination together with the ultrasound transducer. The glove comprises a palm portion integral with a thumb finger pocket, an index finger pocket and one or more additional finger pockets, and the glove wall further defines an ultrasound transducer pocket integral with the palm portion and communicating with the inside of the glove, the preferably elongated transducer pocket extends either i) between the thumb finger pocket and the index finger pocket or ii) being arrangeable to extend between the thumb finger pocket and the index finger pocket.

Preferably, the transducer pocket has a length equal to or longer than the index finger pocket, to receive the full length of the transducer part of the transducer, with a handle part thereof being inside the palm portion in direct contact with the palm of the wearer of the glove; more preferably, where the glove body has five finger pockets, each configured for receiving a respective finger of the hand, the length of the transducer pocket conveniently may be longer than the length of any of the finger pockets. The length of the pockets is determined as the distance between the tip of the pocket and the root of the pocket where the pocket extends from the palm portion; where applicable, this length may for each pocket be determined as the average of that length, measured along the transition between the pocket and the palm portion.

Further embodiments of the invention are defined in the dependent claims, the objects of which will appear from the following.

### DESCRIPTION OF THE DRAWINGS

A presently preferred embodiment of the invention will now be described with reference to the appended drawings; the following description is not intended to limit the scope of the present invention which is defined by the claims.

FIG. 1 shows by way of example and ultrasound examination kit,

FIG. 2a shows an embodiment of a glove according to the invention,

FIG. 2b shows another embodiment of a glove according to the invention,

FIG. 3 shows a mold for forming a third embodiment of a glove according to the invention, where an ultrasound transducer pocket is arrangeable to extend between the thumb finger and the index finger of a person wearing the glove, and

FIGS. 4a and 4b show embodiments of the glove exhibiting a mitten-like structure.

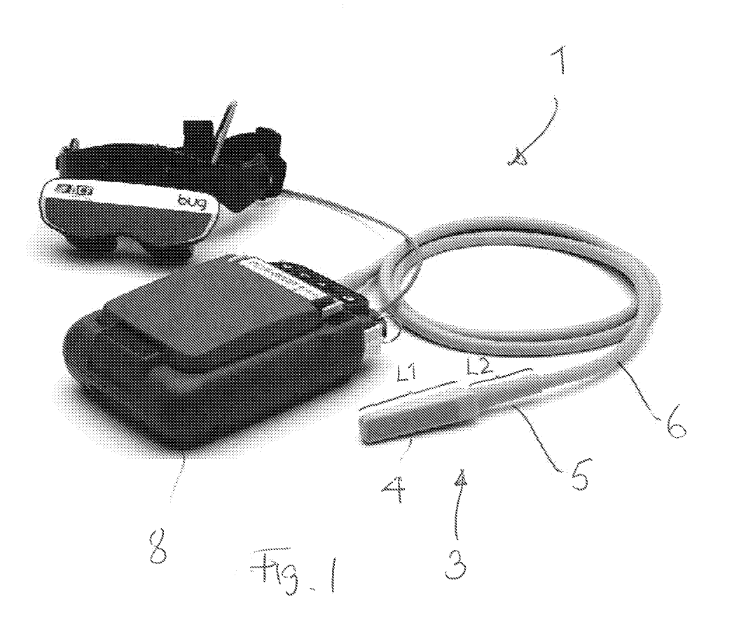

FIG. 1 shows an example of a known ultrasound veterinary examination kit 1 (manufactured in this case by BCF Technology Inc.), for insertion into the rectal canal of a large animal, such as a cow, and including a data processing unit 8 connected by cable 6 to a possibly elongated ultrasound transducer 3 having a transducer part 4 and a handle part 5. Conventionally, a veterinarian, after having inserted his hand into a known examination glove, such as shown by way of example in KR 20-2012-0002725, will hold onto the handle part 5 with his gloved hand and insert his arm and hand with the transducer 3 and the cable 6 deep into the rectal canal of the large animal to be examined. He will then rotate his hand as required, to obtain a desired ultrasound imaging. Often, the length L1 of the transducer part 4 is longer than the length of a finger of a person.

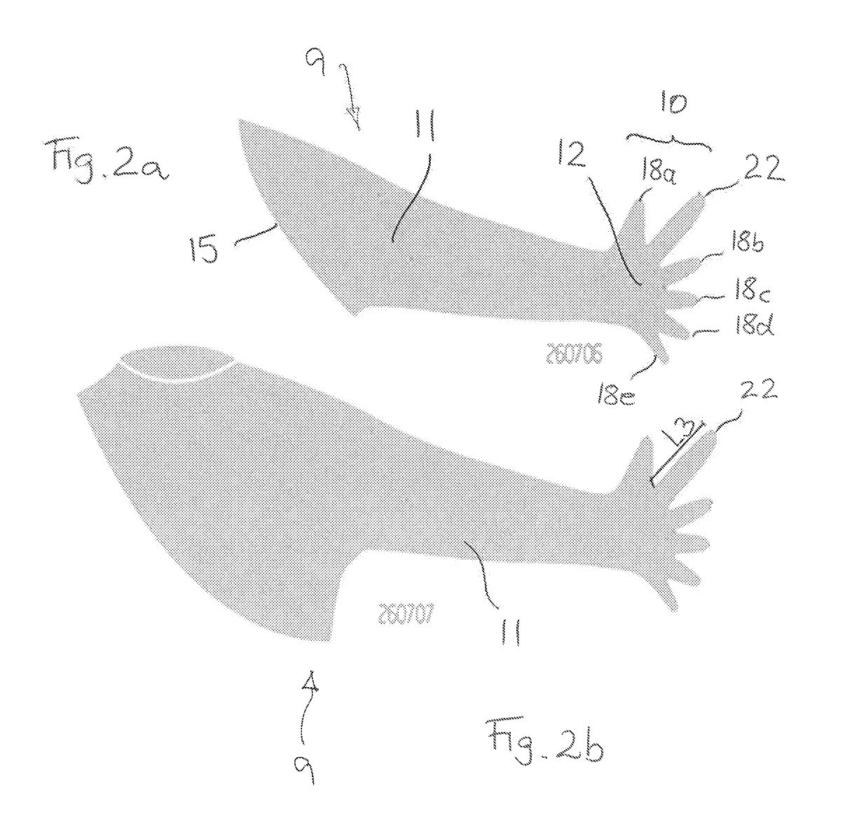

FIG. 2a is a plane view of an improved veterinary examination glove 9 according to the present invention and having a smooth and soft outer surface formed by a liquid tight enclosure or wall 15 defining the inside of the glove 9. The shown hand receiving glove 9 is a preferred embodiment that includes an integral tubular sleeve portion 11 configured to extend up to the shoulder of a person wearing the glove 9. FIG. 2b shows an alternative embodiment, similar to FIG. 2a but having a further extension configured to be wearable also on the upper part of the torso a person.

The glove 9 generally may be formed from a sheet-material, such as a polyethylene sheet material, constituting the fluid tight glove wall 15, and includes a glove body 10 with a palm portion 12 receiving the palm part of the wearer's hand and being integral with finger pockets communicating with the interior of the palm portion 12. The finger pockets include a thumb finger pocket 18a, an index finger pocket 18b and additional finger pockets 18c, 18d, 18e. A further integral transducer pocket 22 of length L3 communicates with the inside of the palm portion 12, and extends between the thumb finger pocket 18a and the index finger pocket 18b. In a variant, not shown, the transducer pocket 22 may extend at least partially along the length of the palm portion 12, still inside the glove 9.

Preferably, the glove wall 15 also defines an elongated sleeve portion 11 integral with the palm portion 12 for receiving at least a portion of an arm of the person performing the examination.

Preferably also, the glove body 10 is flat and made of layers fused along their edges which together define the wall 15.

In use the veterinarian will normally apply an ultrasound lubricant onto the transducer 4, and he then inserts the transducer 3 into the glove 9 shown in FIG. 2a/b via its open end, at the end of sleeve 11. It may be preferred to invert the glove 9 somehow, to allow for the transducer 3 to be easily inserted into the transducer pocket 22 that communicates with the palm portion 12, with the cable 6 trailing the transducer 3 along the length of the sleeve 9 to be connected with the processing unit 8.

The transducer 3 frequently is of relatively large length L1+L2, as shown in FIG. 1, such as in the order of 25 cm, with the length of the handle part 5 being designated L2 in the figure. The handle part 5 is the portion of the transducer 3 that a user will hold around with his index finger and his thumb finger during the examination/ultrasound scanning procedure; the length L3 of the transducer pocket 22 is preferably selected to be longer than the length of any of the finger pockets 18a-18e such that the transducer 3 can be inserted into the glove 9 with its elongated transducer part 4, which normally is longer than the length of a person's finger, being fully received in the transducer pocket 22, with the handle 5 extending fully or substantially in the palm portion 12. This allows the user to firmly grip onto the handle part 5 without his grip interfering with the transducer part 4.

Manufacture of the glove 9 shown in FIG. 2a/2b may be by fusing two sheets of a flexible plastics material along lines delimiting the palm portion 12, the finger pockets 18a-18e and the transducer pocket 22 located between the thumb pocket 18a and the index finger pocket 18b, and then punching out the glove 9 from the thus joined sheets. The basic steps in such a process is described in EP 1 182 942.

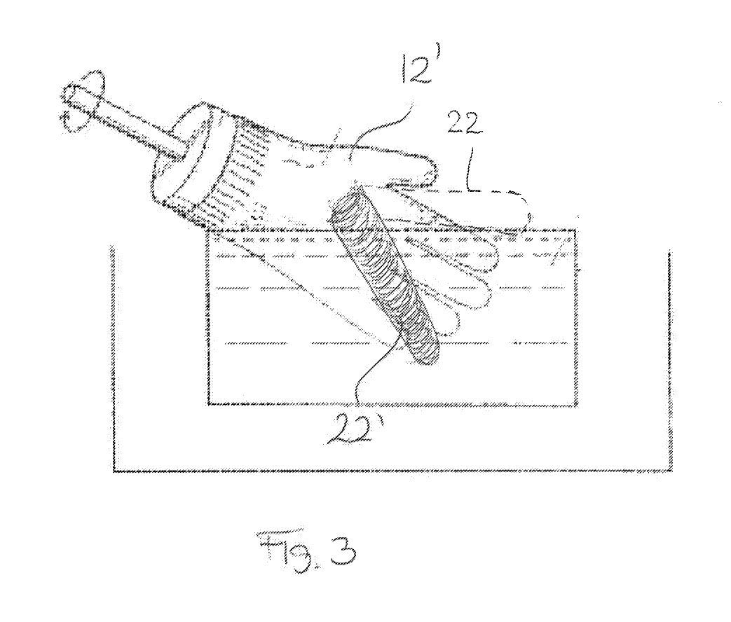

Alternatively, the glove 9 may be formed in a dipping process using a mold as schematically shown in FIG. 3 which is dipped in a liquid material, such as synthetic or natural rubber, which hardens to form the glove 9. As will be seen, in this process a mold part 22′ located so as to project outwards from the palm portion 12′ forms the aforementioned transducer pocket 22 which is this case is capable of being arranged to extend between the thumb finger pocket 18a and the index finger pocket 18b in use of the glove 9, as shown in broken line in FIG. 3, such that the wearer of the glove 9 can hold the handle part 5 of the transducer 3 between his thumb finger and his index finger.

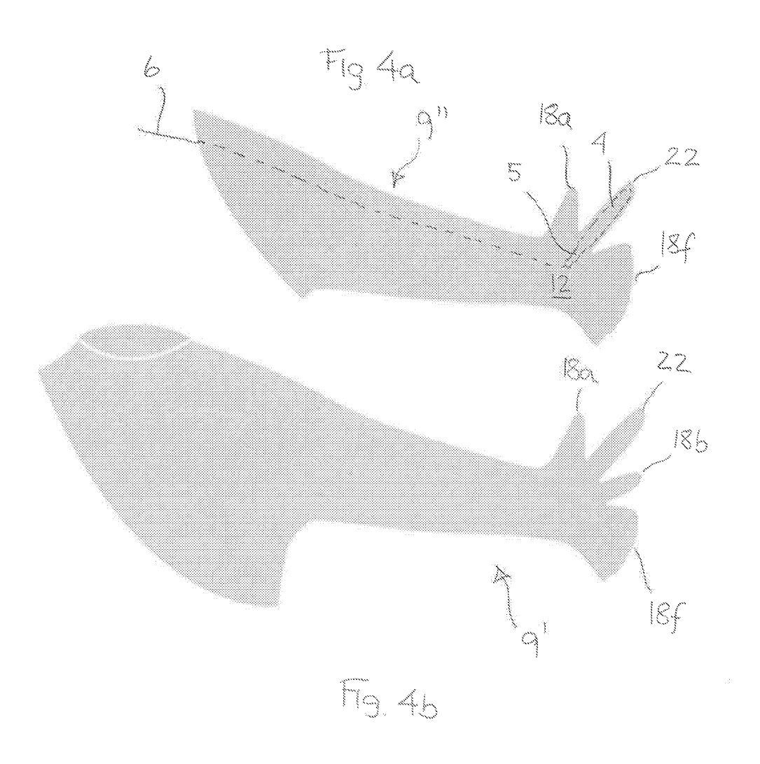

While it is normally preferred to form the glove 9 with five finger pockets it lies within the scope of the appended claims to form the glove 9′ as a mitten or mitten-like structure with one pocket 18f suitable for receiving more than one finger, such as the three fingers following the index finger, and with the thumb finger pocket 18a and index finger pocket 18b as shown in FIG. 4b; a similar such structure is shown in DE 1 038 712. Another embodiment, included herein to show a broadest teaching of the invention, is shown in FIG. 4a where a single pocket 18f received all four fingers 18b-18e next to the thumb finger.

FIG. 4a has also been included herein to show how the transducer 3 generally and preferably is received within the glove in the aforementioned embodiments, i.e. with the transducer part 4 in the aforementioned additional transducer pocket 22, the handle part 5 in the palm portion 12, and with the cable 6 extending along the length of the sleeve 11. While the glove 9 may have a dedicated internal channel (not shown) in which the transducer 3 and cable 6 is run to the transducer pocket 22 without contacting the skin of the wearer such a solution is currently not preferred.

1. A veterinary glove (9) for use in rectal ultrasound examination of a large animal by means of an ultrasound transducer, the glove (9) having a fluid tight glove wall (15) defining a hand receiving glove body (10) for receiving a hand of a person performing the examination together with the ultrasound transducer, characterized in that
the glove (9) comprises a palm portion (12) integral with a thumb finger pocket (18a), an index finger pocket (18b) and one or more additional finger pockets (18c, 18d, 18e),
the glove wall (15) further defining an ultrasound transducer pocket (22) integral with the palm portion (12) and communicating with the inside of the glove (9), the ultrasound transducer pocket (22) extending either
i) between the thumb finger pocket (18a) and the index finger pocket (18b) or
ii) being arrangeable to extend between the thumb finger pocket (18a) and the index finger pocket (18b).

2. The glove of claim 1, the transducer pocket (22) having a length equal to or longer than the index finger pocket (18b).

3. The glove of claim 1, the glove body (10) having five of the finger pockets (18a-18e), each configured for receiving a respective finger of the hand.

4. The glove according to claim 1, the transducer pocket (22) having a length longer than any of the finger pockets (18a-18e).

5. The glove according to claim 1, the glove wall (15) also defining an elongated sleeve portion (11) integral with the palm portion (12), for receiving at least a portion of an arm of the person performing the examination.

6. The glove according to claim 1, the glove body (10) being flat and made of layers fused along their edges to define together the wall (15).

7. The glove according to claim 1, the glove wall being formed from sheets of polyethylene material and having the elongated transducer pocket extending between the thumb finger pocket and the index finger pocket, or the glove wall being formed of a latex material in a molding operation and having the elongated transducer pocket arrangeable to extend between the thumb finger pocket and the index finger pocket.

8. A veterinary implement for rectal ultrasound examination of a large animal, including a glove and an elongated ultrasound transducer, the glove having a fluid tight glove wall defining a hand receiving glove body (10) for receiving the hand of a person performing the examination, the hand receiving glove body (10) including a palm portion integral with a thumb finger pocket, an index finger pocket and one or more additional finger pockets, the glove wall also defining an elongated transducer pocket integral with the palm portion and communicating with the interior of the palm portion, the elongated transducer pocket i) extending between the thumb finger pocket and the index finger pocket or ii) being arrangeable to extend between the thumb finger pocket and the index finger pocket, the elongated ultrasound transducer extending in the transducer pocket.

9. The implement according to claim 8, the transducer pocket having a length equal to or longer than the index finger pocket.

10. The implement according to claim 8, the glove body having five finger pockets, for receiving a respective finger of a person performing the examination.

11. A method of preparing the implement of claim 8, comprising the steps of i) applying an ultrasound lubricant to the transducer, and ii) inserting the transducer into the transducer pocket via the palm portion, with transducer connecting cables extending out from the glove.
